(19) United States
(12) Patent Application Publication
(10) Pub. No.: US 2017/0079543 A1
(43) Pub. Date: Mar. 23, 2017

(54) Imaging compatible electrode-set for measurement of body electrical signals and methods for fabricating the same using ink-jet printing

(71) Applicant: NeuroRex Inc, Houston, TX (US)

(72) Inventor: Seyedhesam Sadeghian-Motahar, Houston, TX (US)

(21) Appl. No.: 15/268,600

(22) Filed: Sep. 18, 2016

Related U.S. Application Data
(60) Provisional application No. 62/220,876, filed on Sep. 18, 2015.

(51) Int. Cl. A61B 5/0478; A61B 5/0424; A61B 5/0492; B41J 2/01

(52) U.S. Cl. CPC A61B 5/0478; B41J 2/01; A61B 5/0424; A61B 5/0492; A61B 2562/18

(57) ABSTRACT

This invention relates to devices and methods for measuring and/or recording electrical signals from a body, particularly to devices and methods for recording electrical signals from a body while inside a medical imaging device, such as magnetic field scanner, such as a magnetic resonance imaging (MRI) scanner, or a computerized tomography (CT) scanner, and more particularly to devices, such as headpieces with electrodes and conductive pathways, and methods for synthesis of conductive inks used to fabricate the devices, such as with inkjet printer technology. Inkjet compatible inks may be utilized that employ nanoparticle solutions or metalorganic decomposition to generate metallic depositions, such as of silver, without sintering or other secondary processing in predetermined, including customized, layouts.

### CROSS-REFERENCE TO RELATED APPLICATIONS

This application claims the benefit and priority of U.S. provisional patent application Ser. No. 62/220,876, filed Sep. 18, 2015, entitled “Magnetic resonance imaging compatible lead-cap for concurrent recording of dense array electroencephalogram (EEG) at high field MRI”, the contents of which is hereby incorporated by reference in its entirety.

### FIELD OF THE INVENTION

This invention relates to devices and methods for measuring and/or recording electrical signals from a body, particularly to devices and methods for recording electrical signals from a body while inside a medical imaging device, such as magnetic field scanner, such as a magnetic resonance imaging (MRI) scanner, or a computerized tomography (CT) scanner, and more particularly to devices, such as headpieces with electrodes and conductive pathways, and methods for synthesis of conductive inks used to fabricate the devices, such as with inkjet printer technology.

### BACKGROUND OF THE INVENTION

Typical electrodes for electroencephalogram (EEG) recordings are cup or disc electrodes made out of brass with copper leads. They are often reused after each recording session hence if not properly cleaned and disinfected; it will introduce concerns about cross-infections within Emergency Department (ED) and Intensive Care Unit (ICU). They also require technical expertise to apply and maintain. For example, typically a technologist takes measurements of the head, for example, to measure the distance from Nasion to Inion, left ear preaurical point to right ear preaurical point and head circumference, to setup 10-20 electrode placements on a subject's head (an international positioning system for diagnostic EEG). This head measurement and electrode setup is time consuming and typically requires significant training.

The cup or disc electrodes (conventional EEG electrodes) also cause artifacts on CT and MRI scanners so EEG recording is frequently interrupted for patient population in ED/ICU undergoing other neuroimaging procedures such as CT/MRI imaging.

In current MRI compatible EEG systems, the electrodes are connected to EEG amplifier via metallic wires that interfere with the magnetic field within the MR scanners, increasing the risk of tissue heating, producing errors and artifacts in the sensitive dense EEG recordings and MR images.

Of the 136 million patient visits in U.S. hospital emergency departments (ED) annually, as many as 10% of patients in EDs have altered mental status for whom electroencephalogram (EEG) is among the first-line tests used to diagnose their conditions. The use of EEG in the ED and Intensive Care Unit (ICU) faces numerous obstacles due to lack of an optimal EEG electrode-set and the shortages of qualified professionals to administer EEGs. Often used electrodes for EEG are disc or cup electrodes which require technical expertise to apply and suffer from lengthy set-up times and are not optimal for rapid application of EEG in ED/ICU. Rapid assessment of EEG is crucial for effective therapy of patients with Status epilepticus (SE)—which is a prolonged seizure(s) during which the patient has incomplete recovery of consciousness and is considered a medical emergency with a mortality rate of 20% and an annual incidence of greater than 100,000 cases in the United States alone. Delayed recognition or treatment can result in a worse neurologic outcome for SE patients. Other electrode-sets commonly used are EEG electrodes embedded in electrode-caps and nets. These may allow for quick placement without head measurement, but may not be suitable for use in ICU in situations, for example, involving head wounds and skin infections and concerns about cross-infection. The alternative single-use disposable stainless steel needle electrodes at ICU pose a risk for needle-stick injury to ICU personnel if dislodged, and may generally not be suited for prolonged recordings. Needle electrodes also cause some artifacts on CT and are not MRI compatible.

InkCap (U.S. Patent Application Publication US20140249612 A1): is an EEG cap designed for use in high field MR. The conductive ink for making electrodes and leads on “InkCap” is used to reduce Radio-Frequency heating. An electrode on the InkCap is a composite of blended silver/carbon ink printed on a polymer film and Ag/AgCl printed on a vinyl paper. However, this cap used only 32 channels and EEG caps less than 64 channels have negligible influence on the MRI signals. InkCap is fabricated using screen-printing. There is a draw back with screen-printing as it involves printing the design on a transparency film and applying a photo emulsion to create a positive of the design (stencil) and drying the screen in a dark location presents a chance of over exposure to light which ruins the stencil. Typically, following screen-printing, the screen is preceded with heating at curing temperatures above 120 Celsius which prevents printing and their usages on low-temperature substrates such as Polyester films and other plastics.

U.S. Patent Application Publication 20100041962 A1: an EEG head-set screen-printed on a flexible substrate however this head-set only covers subject's forehead and hairless region of the head and since it's not a full head montage, its therapeutic benefits are limited and cannot be used for epilepsy diagnosis (requires full head montage).

### SUMMARY OF THE INVENTION

This invention relates to devices and methods for measuring and/or recording electrical signals from a body, particularly to devices and methods for recording electrical signals from a body while inside a medical imaging device, such as magnetic field scanner, such as a magnetic resonance imaging (MRI) scanner, or a computerized tomography (CT) scanner, and more particularly to devices, such as headpieces with electrodes and conductive pathways, and methods for synthesis of conductive inks used to fabricate the devices, such as with inkjet printer technology.

In general, it may be desirable to manufacture devices for measuring electrical signals from a body, which may generally refer to any appropriate subject for measurement such as a human body or portions thereof (or equivalent portions of an animal), which may include, but are not limited to, the head or portion of, the torso or portion of, or any other appropriate portion of a human body, from disposable and/or otherwise low-cost materials. In clinical applications, particular in emergency situations and similar areas, utilizing disposable items may be desirable to aid in rapid treatment or assessment, such as, for example, without concerns about recovering and sterilizing items, cross-contamination between patients, or applying or reapplying materials such as adhesives or conductive fluids between patients. Further in general, it may be desirable to utilize devices for measuring electrical signals from a body that utilize a minimal amount of metallic or other materials that may interfere with MRI or CT devices, such as, for example, significant quantities or interfering configurations of metals, in particular ferrous metals.

In one aspect of the invention, a device for measuring and/or recording electrical signals from a body, such as, for example, from the human head or similar bodily structures, may generally include a plurality of spatially arranged electrodes which may contact particular locations on a body to detect electrical activity, and which may connect to a signal measuring/recording device via conductive electrical connection(s). In some exemplary embodiments, the spatially arranged electrodes may be attached or formed onto a non-conductive or electrically insulating substrate which may be flexible or deformable. The electrodes may further be connected to electrically conductive pathways or other conductive connections, which may, for example, also be attached or formed onto the substrate in an ordered manner.

In some exemplary embodiments, the substrate may be a generally flat or substantially planar material on which conductive materials may be deposited in a predetermined fashion to form conductive pathways (traces) and/or electrodes, such as in a substantially flat or thin layer deposition. In general, flat or planar (or substantially so), may be understood to include any materials where the surface is primarily smooth, flat and/or generally uninterrupted by protrusions or other formations, and having a material thickness much less than the surface area, such as created by the length and width, such as with general polymer films, paper, foil or other similar materials. For example, and without any limitation, substantially planar or flat materials may take the form of a thin film, such as a film having a thickness of about 2-100 millimeters, and having dimensions on the order of normal sheets of paper, such as letter, legal, A4 or other standard dimensions. The disposition of conductive pathways and/or electrodes may, for example, be designed or optimized to aid in applying the substrate, either whole or after excess material has been removed, to a body having a three dimensional surface, such as the human head. The relatively small quantities of material utilized to produce thin layers of conductive material, such as through controlled deposition of conductive ink using inkjet printing (which may produce, for example, lines as small as 20-100 microns) may, for example, be desirable to reduce interference from such conductive materials in the operation of MRI or CT devices. Additionally, non-ferrous conductive materials, such as silver and carbon-based conductive materials, may be utilized for their reduced interactions with MRI or CT devices. This may be desirable such that devices may be left in place on a patient, for example, during examination utilizing MRI or CT without loss of time due to removal and/or application of devices before or after imaging (or potentially loss of measurements during that time).

Additional items or features, such as insulating or protective layers, contact aids, adhesives, conductive gels or other conductivity enhancing materials, may also be applied to the substrate.

In some embodiments, the substrate may be printed with a predetermined set of electrodes and conductive pathways utilizing electrically conductive materials in the form of ink, such as with ink compatible with inkjet printing. For example, an appropriately formulated ink such as metalorganic ink, may be utilized with an inkjet or similar printer to print the predetermined formation of electrodes and conductive pathways onto a substrate. This may be desirable as inkjet printers are readily accessible and easily utilized with computer design software to create desired depositions of ink. Further, appropriate inks, such as inks which generate conductive pathways without the use of sintering or other high temperature operations, may be desirable as they may enable the use of a broader range of high temperature-sensitive substrate materials, such as most common polymers and cellusolic materials.

In some exemplary embodiments, measurements of a subject or patient may be utilized to generate a custom printed substrate. For example, desired locations for electrode placements on a body may be marked and/or otherwise mapped and converted to a two dimensional layout, such as with computer-aided design, to design a pattern of electrode and trace placements for printing or otherwise depositing onto a substrate. The resulting substrate may then be cut to remove excess material, if necessary, and attached to the body at the predetermined locations.

In another aspect, a device for measuring electrical signals from a body may include a special cap based on conductive yarns (e.g. carbon nanotube yarn) that incorporates electroencephalogram (EEG) electrodes, to record high quality EEG inside MRI/fMRI/MEG scanner.

In some embodiments, this electrode-set or head-piece may come in form factor of a cap, which replaces metallic wires with conductive fabric embedded onto cap (such as carbon nanotube yarn), which may 1) decrease the risk of tissue heating and minimize errors arising from interference with the magnetic field within MR scanner, 2) improve wearability of head-piece using fabric based leads out of conductive yarn.

Conductive fabric embedded onto cap (such as carbon nanotube yarn) may reduce tissue heating from induced Radio-Frequency, improves MRI data quality and EEG data integrity inside MR scanner. Conductive fabric embedded onto cap (such as carbon nanotube yarn) also used for concurrent recording of EEG inside Magnetoencephalogram (MEG) scanners.

The present invention together with the above and other advantages may best be understood from the following detailed description of the embodiments of the invention illustrated in the drawings.

### BRIEF DESCRIPTION OF THE FIGURES

FIG. 1 illustrates an embodiment of a bio-potential sensor set on a substrate;

FIG. 1a illustrates an example of a bio-potential sensor set on a flexible substrate;

FIG. 1b illustrates the application of a flexible substrate with a bio-potential set applied to an object and connected to a medical device;

FIGS. 2 and 2a illustrate embodiments of electrode connecting pieces with conductive fluid or gel reservoirs;

FIG. 3 illustrates MRI-compatibility testing of inkjet printed electrode set and traditional electrode set using balloon phantoms; and

FIG. 4 illustrates preliminary EEG data using inkjet printed electrode set and traditional electrode set on a human subject.

### DETAILED DESCRIPTION OF THE INVENTION

The detailed description set forth below is intended as a description of the presently exemplified devices, methods and materials provided in accordance with aspects of the present invention, and it is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

This invention relates to devices and methods for measuring and/or recording electrical signals from a body, particularly to devices and methods for recording electrical signals from a body while inside a medical imaging device, such as magnetic field scanner, such as a magnetic resonance imaging (MRI) scanner, or a computerized tomography (CT) scanner, and more particularly to devices, such as headpieces with electrodes and conductive pathways, and methods for synthesis of conductive inks used to fabricate the devices, such as with inkjet printer technology.

Simultaneous EEG-fMRI recording is a technique of importance in neuroscience that studies spatiotemporal dynamics of brain function by combining the high temporal resolution of EEG with the high spatial resolution of the MRI in images acquired in a single measurement session. EEG-fMRI recording is used for localizing epileptic focus, determining sources of event related potentials or correlating brain rhythms with hemodynamic activities. High density EEG systems (64 channels or more) provide higher spatial resolution for measuring the scalp EEG signal and improve the accuracy of localizing cortical responses.

In general, it may be desirable to manufacture devices for measuring electrical signals from a body from disposable and/or otherwise low-cost materials. In clinical applications, particular in emergency situations and similar areas, utilizing disposable items may be desirable to aid in rapid treatment or assessment, such as, for example, without concerns about recovering and sterilizing items, cross-contamination between patients, or applying or reapplying materials such as adhesives or conductive fluids between patients.

Low-cost disposable EEG electrode set devices with MRI or CT compatibility may be designed to be applied by someone without specialized training, targeted for rapid Electroencephalography (EEG) in clinical emergency departments (ED to achieve earlier detection and treatment of Non-Convulsive Status Epilepticus (NCSE) and other seizure-related conditions. Configurations may be utilized, for example and without limitation, in routine EEG, long-term epilepsy studies, and sleep studies, mobile applications in ambulances or battle fields, Electrocardiogram (EKG) and electromyogram (EMG) applications.

Further in general, it may be desirable to utilize devices for measuring electrical signals from a body that utilize a minimal amount of metallic or other materials that may interfere with MRI or CT devices, such as, for example, significant quantities or interfering configurations of metals, in particular ferrous metals.

In one aspect of the invention, a device for measuring and/or recording electrical signals from a body, such as from the human head or similar bodily structures, may generally include a plurality of spatially arranged electrodes which may contact particular locations on a body to detect electrical activity, and which may connect to a signal measuring/recording device via conductive electrical connection(s).

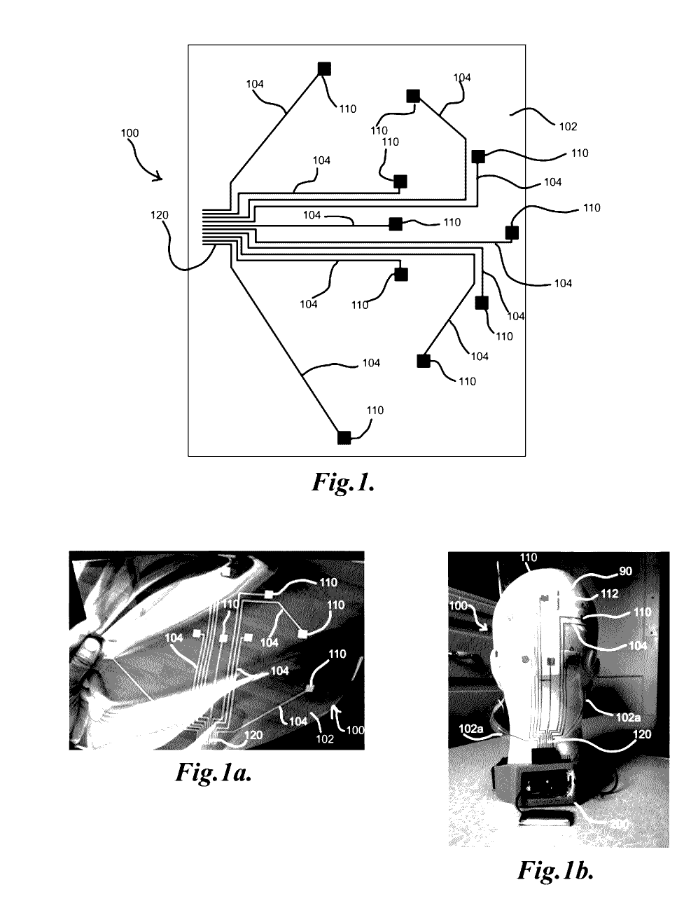

FIG. 1 illustrates an example of a spatially arranged set of electrodes 110 on a substrate 102 to form an electrode set 100. In some exemplary embodiments, the spatially arranged electrodes may be attached or formed onto a non-conductive or electrically insulating substrate which may be flexible or deformable, as illustrated with substrate 102 in FIGS. 1 and 1a. The electrodes may further be connected to electrically conductive pathways or other conductive connections, which may, for example, also be attached or formed onto the substrate in an ordered manner, as shown with traces 104 deposited onto substrate 102 connecting electrodes 110 to an output connector 120.

In some exemplary embodiments, the substrate may be a generally flat or planar material on which conductive materials may be deposited in a predetermined fashion to form conductive pathways (traces) and/or electrodes, such as in a substantially flat or thin layer deposition. The disposition of conductive pathways and/or electrodes may, for example, be designed or optimized to aid in applying the substrate, either whole or after excess material has been removed, to a body or head having a three dimensional surface, such as the human head. FIG. lb illustrates an example of a human head shaped object 90 with an electrode set 100 attached to the surface at predetermined points with electrodes 110 and also connected to a measurement device 200, such as an EEG amplifier, via output connector 120. The excess substrate material may be removed to leave strips of material 102a that carry, for example, individual traces 104 and electrodes 110, such that they may, for example, be individually placed and/or handled.

Any appropriate material may be utilized for the substrate and they may include, but are not limited to, polymer films or sheets, such as polyethylene, polyethylene terephthalate (PET) or variations such as biaxially oriented PET (BoPET), polypropylene, polyimide, polyethylene-naphthalate, fluorinated polymers (e.g. Teflon, PTFE, PFA, FEP, etc.) polyester, silicone, polyurethane, or vinyl; paper; foils; medical foam; non-woven fiber or filament sheets and/or any other appropriate material or combination thereof. In general, it may be desirable to select a non-conductive substrate material such that it does not interfere or cause short circuiting of the conductive pathways. The substrate may also be any appropriate thickness, such as, for example, about 2 to 10 millimeters in thickness. The substrate should generally be of a thickness to retain desirable flexibility, such as necessary to form around a three dimensional object such as a human head, and also be of an appropriate thickness for the desired deposition technique of the conductive pathways and electrodes.

In some exemplary embodiments, measurements of a subject or patient may be utilized to generate a custom printed substrate. For example, desired locations for electrode placements on a body or head may be marked and/or otherwise mapped and converted to a two dimensional layout, such as with computer-aided design, to design a pattern of electrode and trace placements for printing or otherwise depositing onto a substrate. The resulting substrate may then be cut to remove excess material, if necessary, and attached to the body or head at the predetermined locations. Which allows rapid deployment of head-piece to subject's head by a non-trained individual or an individual with minimal training (non-technologists).

The relatively small quantities of material utilized to produce thin layers of conductive material may, for example, be desirable to reduce interference from such conductive materials in the operation of MRI or CT devices. Additionally, non-ferrous conductive materials, such as silver and carbon-based conductive materials, may be utilized for their reduced interactions with MRI or CT devices. This may be desirable such that devices may be left in place on a patient, for example, during examination utilizing MRI or CT without loss of time due to removal and/or application of devices before or after imaging (or potentially loss of measurements during that time).

Additional items or features, such as insulating or protective layers, contact aids, adhesives, conductive gels or other conductivity enhancing materials, may also be applied to the substrate. For example, a protective layer may be applied over the conductive pathways and/or electrodes such that the material is not disrupted or subject to environmental factors or agents which may cause corrosion, short circuits or other disruptions. In general, protective layers may be removable or not present in areas where electrical connections are to be made, such as at the contact points of the electrodes 110 for contacting the body or the contacts of the output connection 120.

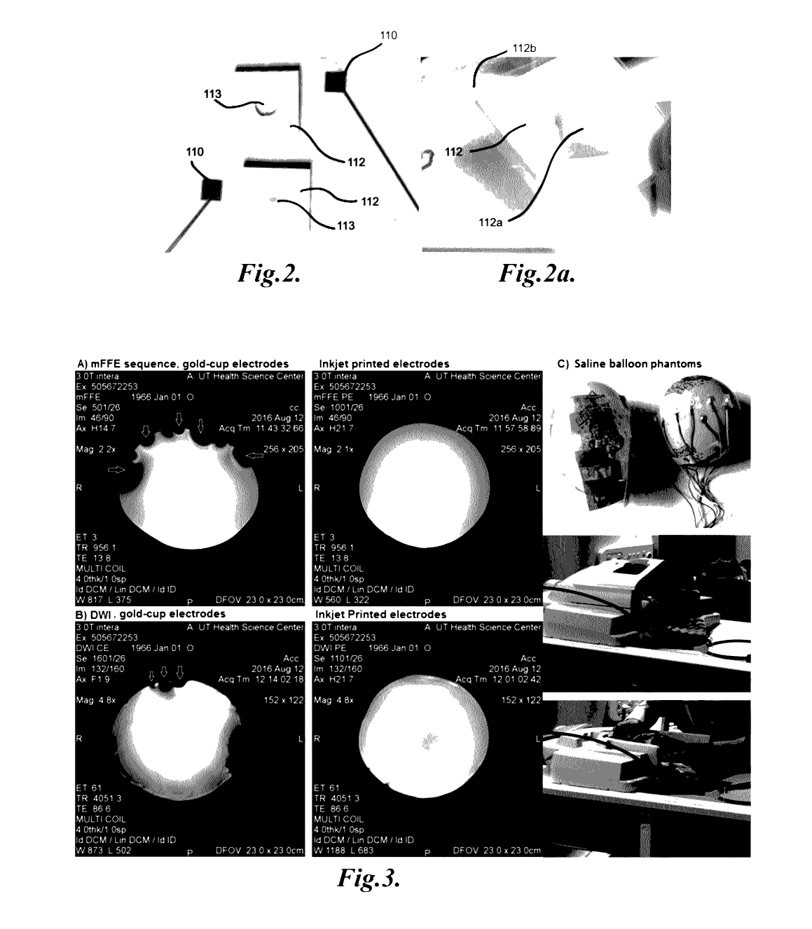

In general, certain body parts may have hair or other obstacles for electrical contact between the electrode 110 and the desired surface. In some embodiments, a soft foam soaked with fluid or conductive gel may be utilized as a conductive bridge between the surface and the electrode 110, such that there may be low resistance/impedance between the body 90 and electrode 110. FIG. 2 illustrates a conductive fluid or gel reservoir filled in a cavity 113 formed in a connector 112. The connector 112 may generally be prefilled with a conductive fluid or gel and applied to the body at a point of interest. In general, the connector 112 may include an adhesive or other feature for attaching to the body and/or to an electrode 110 or surrounding substrate 102, such that the conductive fluid or gel bridges the body and the electrode 110. The connectors 112 may also feature, for example, a penetrating portion, such as penetrator 112a, which may be utilized to pierce through hair or other obstacles, such as to contact the scalp. The penetrator 112a may, for example, be porous such that the conductive fluid or gel may permeate and carry through the penetrator 112a to provide a conductive path between the surface and the electrode 110, as illustrated in FIG. 2a. The connectors 112 may be preapplied to the body 90 or to the substrate 102, such as with peel-off backing for adhesive attachments, as shown with backing 112b. The conductive fluid or gel may, for example, include Ag—AgCl gel, NaCl solution/gel, and/or any other appropriate conductive fluid or gel.

In some embodiments, the substrate may be printed with a predetermined set of electrodes and conductive pathways utilizing electrically conductive materials in the form of ink. For example, an appropriately formulated ink, such as metalorganic ink, may be utilized with an inkjet or similar printer to print the predetermined formation of electrodes and conductive pathways onto a substrate. Deposition of metal ink to produce the conductive pathways and/or electrodes on a substrate through inkjet printing may be desirable, such as over other deposition methods such as screen-printing, such as by (i) enabling easy customization and robust fabrication of the electrode positions and/or conductive pathways on a substrate with computer software, such as for generating a headpiece (ii) efficient handling of expensive materials, and (iii) enabling controlled deposition of metal lines and grids by printing very fine lines as small as, for example, 20-30 μm wide or smaller as technology may allow. Further, appropriate inks, such as inks which generate conductive pathways without the use of sintering or other high temperature operations, may be desirable as they may enable the use of a broader range of high temperature-sensitive substrate materials, such as most common polymers and cellusolic materials.

In some embodiments, the ink for use with an inkjet printing method for producing an electrode set includes a liquid (water or an organic solvent), which may generally determine the basic properties of the ink, and dispersed metal nanoparticles or dissolved metal precursors of conductive metals such as Silver (Ag), Copper (Cu), Gold (Au), Aluminum (Al), and/or any other appropriate metal or combination thereof. Viscosity, surface tension, and wettability are characteristics of the formulated conductive ink which may generally be adjusted to meet criteria for inkjet printing since they may, for example, affect printing quality, drop size, drop placement accuracy, satellite formation, wetting of the substrate, etc.

In some embodiments of a formulated ink, the ink viscosity may be in the range of 8-15 cP, surface tension, in the range of 25-35 dyne/cm (which may be in the typical value ranges for industrial printheads). The formulation may also include a binder, such as a thermoset or thermoplastic resin, which may improve adhesion of the printed traces and/or electrodes to the substrate.

In some embodiments, nanoparticle solutions may be utilized in an ink formulation to produce conductive depositions.

In some exemplary embodiments, the formulated ink may be based on metalorganic decomposition (MOD), which generally employs, for example, metal-organic (MO) precursors with a reducing agent that provides a counter-ion for reaction with the MO precursor at an activating temperature to produce pure or highly pure metal deposits on the substrate. For example, the chemical formulation may include: a solvent which provides the wetting properties suitable for inkjet printing, organometallic precursors and a reducing agent that is in liquid phase, such as at room temperature. Elevated temperature may then be used to activate the MOD ink, such as by heating the substrate (e.g. in the range of 80-110° C.) which may then activate the catalytic component of the ink. The ink may then be converted to a metallic deposition upon contact with the heated substrate, and may thus require no further processing to form conductive pathways.

In some exemplary embodiments, the conductive pathways and/or the electrodes may be formed from silver or silver containing materials. Silver is generally non-toxic and biocompatible with good conductivity and some resistance to corrosion. Silver is commonly used in electrodes, such as in the form of silver-silver chloride electrodes.

In another aspect, application of this technology may enhance the data quality that helps understand the spatiotemporal processes of complex large-scale brain network and contributes to many aspects of cognitive neuroscience, sleep studies, evoked potential studies and treatment of many neural pathologies particularly epilepsy to improve epilepsy surgical workup process and outcomes.

Lead-cap may be fabricated by sewing conductive yarns such as carbon nanotube yarn e.g. cYarn™ by Lintec of America onto BrainCap MR Model (BrainVision, Morrisville, N.C.) via commercial embroidering machines. Lead-cap assembly with dry-electrodes such as NeuroRex

Spider dry-electrode is done by placing each dry-electrode under the cap fabric and bonding it to the sewn cYarn onto the fabric using conductive silver epoxy paste (Masterbond, Hackensack, N.J.), to incorporate 32, 64, 128, 256 sensor positions according to the expanded 10-20 system. The cap fabric is non-conductive and may include cotton, polyester, nylon and/or any other appropriate material.

### EXAMPLE OF EEG PRELIMINARY DATA

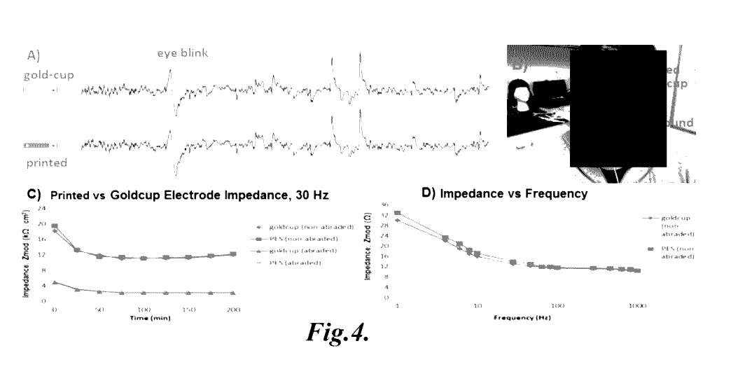

As preliminary means to compare the performance of inkjet printed electrode-set (PES) to conventional EEG electrodes (gold discs with Ten20 conductive paste), simple EEG recording was performed as a proof-of-concept on four subjects, contrasting the performance of PES against that of goldcup electrodes, as illustrated in FIG. 4. EEG was recorded using microEEG amplifier with a 500 Hz sampling frequency, a hardware bandpass filter of 0.1-100 Hz, notch-filter and 0.5 μV amplitude resolution. The presence of awake and resting EEG was observed. The impedance between the gel and counter electrode (impedance spectroscopy is often used to study the electrode-skin interface) was measured on human scalp with potentiostatic EIS using a Gamry FAS2 Femtostat. Qualitative comparison using time-domain and frequency response curve for each channel and across subjects indicated comparable performance between PES and the gold disc electrodes, with similar features of eye blinks, EMG artifacts (jaw clench) and resting state alpha observed in both recordings.

### EXAMPLE OF MRI PRELIMINARY DATA

The effect of inkjet printed electrode-set on MRI data quality was assessed in a Philips 3-Tesla MRI scanner using saline filled balloon phantoms with ink-jetted 10 channel PES on polyimide film and 10 gold-cup electrodes. Two MRI sequences were applied: multi-echo fast field echo (mFFE) and gradient echo diffusion-weighted Echo Planar Imaging (EPI-DWI). PES showed no to very little effect on MRI image quality, as shown in FIG. 3.

### EXAMPLE OF STRATEGY FOR 10-20 PLACEMENT

Strategy for accurate 10-20 placement: a software is developed that takes three measurements: 1) Nasion to Inion 2) left ear preaurical point to right ear's 3) head circumference, and generates a head template with corresponding 10-20 positions (10-20 electrode placements are routinely obtained by technologists from those three measurements). Subsequently the software will convert the marked points representing electrode positions from 3D to a 2D surface for print. Following the print, the traces can be cut using a Xacto pen or die cutter (production level). This head-piece becomes specific to subject's head holding accurate 10-20 positions.

### EXAMPLE OF SYNTHESIS OF ORGANOMETALLIC INK FOR INKJET PRINTING

[(hfac)(1,5-COD)Ag] is silver hexafluoroacetylacetonate cyclooctadiene and propan-2-ol. Varying concentrations of [(hfac)(1,5-COD)Ag] may be dissolved in anhydrous toluene and followed by the addition of propan-2-ol in a volumetric flask to form a series of organometallic:alcohol ink ratios (0.1:0.1 M to ‘excess’ alcohol). The volumetric flask will then be filled with toluene. A chemical reaction for deposition of silver metal onto substrate is carried as the ink hits heated substrate and subsequently reducing to Ag metal.

### EXAMPLE OF FABRICATION OF ELECTRODE-SET OR HEADPIECE

A 10-channel electrode set was inkjet printed on a flexible A4 size polyester film with positions at T5, T6, P3, P4, Pz, O1, Oz, O2, ground and reference interfacing a custom mobile amplifier (ADS1299). The printed side of the flexible substrate sheet was laminated with an insulating layer (e.g. clear thin film or plastic), as shown in FIG. lb.

Electrodes are backed with self-adhered harness, which consists of a double sided medical foam with thicknesses between 1 mm to 5 mm, this foam has a hole (cavity) in center that is filled with EEG ionic gel such as Ag-AgCl (silver-silver chloride) or NaCl (Sodium Chloride) to allow passage of biosensing signals from the scalp to the printed traces. The electrode-set was applied by peeling off the foam backing and adhering to the head of a subject.

A Flat flexible connector (FFC) was included that can terminate in either male pins or females with housing.

The EEG electrode placements can be according to 10-20 international system or Modified Combinatorial Nomenclature (MCN).

The ink-jet printed electrode-set may be cut using Xacto pen or die-cutting machine to wrap around the head (head-piece).

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein (and in particular, the inclusion of any particular embodiment, feature or function within the Abstract or Summary is not intended to limit the scope of the invention to such embodiment, feature or function). Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, “an exemplary embodiment” or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, “in some embodiments” or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

At least a portion of embodiments discussed herein can be implemented using a computer communicatively coupled to a network (for example, the Internet), another computer, or in a standalone computer. As is known to those skilled in the art, a suitable computer can include a central processing unit (“CPU”), at least one read-only memory (“ROM”), at least one random access memory (“RAM”), at least one hard drive (“HD”), and one or more input/output (“I/O”) device(s). The I/O devices can include a keyboard, monitor, printer, electronic pointing device (for example, mouse, trackball, stylist, touch pad, etc.), or the like.

ROM, RAM, and HD are computer memories for storing computer-executable instructions executable by the CPU or capable of being complied or interpreted to be executable by the CPU. Suitable computer-executable instructions may reside on a computer readable medium (e.g., ROM, RAM, and/or HD), hardware circuitry or the like, or any combination thereof. Within this disclosure, the term “computer readable medium” or is not limited to ROM, RAM, and HD and can include any type of data storage medium that can be read by a processor. For example, a computer-readable medium may refer to a data cartridge, a data backup magnetic tape, a floppy diskette, a flash memory drive, an optical data storage drive, a CD-ROM, ROM, RAM, HD, or the like. Software implementing some embodiments disclosed herein can include computer-executable instructions that may reside on a non-transitory computer readable medium (for example, a disk, CD-ROM, a memory, etc.). Alternatively, the computer-executable instructions may be stored as software code components on a direct access storage device array, magnetic tape, floppy diskette, optical storage device, or other appropriate computer-readable medium or storage device.

Any suitable programming language can be used to implement the routines, methods or programs of embodiments of the invention described herein, including the custom script. Other software/hardware/network architectures may be used. For example, the software tools and the custom script may be implemented on one computer or shared/distributed among two or more computers in or across a network. Communications between computers implementing embodiments can be accomplished using any electronic, optical, radio frequency signals, or other suitable methods and tools of communication in compliance with known network protocols. Additionally, any signal arrows in the drawings/figures should be considered only as exemplary, and not limiting, unless otherwise specifically noted. Based on the disclosure and teachings provided herein, a person of ordinary skill in the art will appreciate other ways and/or methods to implement the invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, including the claims that follow, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein, the meaning of “in” includes “in” and “on” unless the context clearly dictates otherwise.

1. A device for measuring electrical activity from a human or mammalian body part comprising:
a flexible substrate, said flexible substrate comprising a substantially planar non-electrically conductive material;
a plurality of electrodes disposed on said flexible substrate at a set of predetermined positions physically corresponding to a set of predetermined positions of interest on a body, said electrodes comprising a thin layer of conductive material;
an interface connector attached to said flexible substrate adapted to electrically communicate with an electrical signal measuring device;
a plurality of conductive traces disposed on said flexible substrate, each of said conductive traces electrically connecting one of said electrodes with an interface connector and comprising a thin layer of conductive material;
wherein said conductive traces are spaced on said flexible substrate at a distance from each other that prevents electrical communication between them.

2. The device of claim 1, wherein said electrodes and conductive traces are disposed on said flexible substrate by inkjet printing of an inkjet-compatible ink formulation containing a metal nanoparticle solution (NP) or a metal organic decomposition formulation (MOD).

3. The device of claim 1, wherein said flexible substrate is selected from the group consisting of a polymer film, paper, foil, foam sheet and a non-woven fiber sheet.

4. The device of claim 1, wherein said flexible substrate is formed to remove excess material from around said electrodes and said conductive traces.

5. The device of claim 3, wherein said flexible substrate comprises a polymer film selected from the group consisting of PET, BoPET, polyimide, polyethylene naphthalate, FEP, PFA, Teflon, PTFE, polyurethane, silicone, polyester, and vinyl.

6. The device of claim 1, further comprising a plurality of penetrators adapted to pierce through hair cover to contact a surface on a human or mammalian body, each of said penetrators being disposed adjacent to one of said electrodes and providing an electrically conductive connection between said electrodes and said surface of a human or mammalian body.

7. The device of claim 6, wherein said penetrators comprise a fluid reservoir prefilled with a conductive fluid or gel.

8. The device of claim 4, wherein said flexible substrate is formed to the shape of a headpiece for EEG.

9. The device of claim 2, wherein said NP solution or MOD formulation generates said electrodes and conductive traces from a metal selected from the group consisting of silver, gold, copper and aluminum.

10. The device of claim 6, wherein each of said penetrators comprise at least one adhesive surface for attaching to said flexible substrate or to said surface of a human or mammalian body.

11. A method for fabricating a set of electrodes on a substrate comprising:
providing a substrate, said substrate being sized and adapted for processing in an inkjet printer;
providing an ink formulation, said ink formulation comprising a metal nanoparticle solution (NP) or a metalorganic decomposition formulation (MOD) and being inkjet printing compatible;
applying said ink formulation to said substrate by inkjet printing in a formation that comprises a plurality of electrodes at predetermined positions and a plurality of traces connecting said electrodes to an area of said substrate designated for connecting to an electrical signal connector, said traces being spaced on said substrate to prevent electrical contact between them; and
activating said ink formulation to produce continuous and conductive metal depositions that form said electrodes and said traces;
wherein said continuous and conductive metal depositions form thin layers of metal on said substrate.

12. The method of claim 11, wherein said activating comprises heating said substrate prior to or during said applying of said ink formulation to activate.

13. The method of claim 11, further comprising forming said substrate to the shape of a headpiece for EEG.

14. The method of claim 13, wherein said forming comprises removing excess material of said substrate.

15. The method of claim 11, further comprising applying a protective layer over said traces.

16. A method for fabricating a custom set of electrodes on a substrate comprising:
determining a set of desired electrode placement locations on a subject by marking three dimensional positions and generating a first data set;
projecting said first data set from three dimensional positions to two dimensional positions;
generating a layout of electrode and trace positions using said two dimensional positions, said traces connecting said electrodes to an area of a substrate designated for connecting to an electrical signal connector and said traces being spaced on said substrate to prevent electrical contact between them;
providing said substrate, said substrate being sized appropriately to encompass said layout and adapted for processing in an inkjet printer;
providing an ink formulation, said ink formulation comprising a metal nanoparticle solution (NP) or a metalorganic decomposition formulation (MOD) and being inkjet printing compatible;
applying said ink formulation to said substrate by inkjet printing; and
activating said ink formulation to produce continuous and conductive metal depositions that form said electrodes and said traces;
wherein said continuous and conductive metal depositions form thin layers of metal on said substrate.

17. The method of claim 16, wherein said activating comprises heating said substrate prior to or during said applying of said ink formulation to activate.

18. The method of claim 16, further comprising forming said substrate to the shape of a headpiece for EEG by removing excess material of said substrate.

19. The method of claim 16, further comprising applying a protective layer over said traces.

20. The method of claim 16, wherein said determining is based on measurements of nasion to inion, left ear preaurical point to right ear preaurical point, head circumference and generation of points based on a standard 10-20 electrode placement.
